(19) United States
(12) Patent Application Publication
(10) Pub. No.: US 2014/0105989 A1
(43) Pub. Date: Apr. 17, 2014

(54) Treatment Methods with Low-Dose, Longer-Acting Formulations of Local Anesthetics and Other Agents

(71) Applicant: Lyotropic Therapeutics, Inc., Ashland, VA (US)

(72) Inventors: David Anderson, Ashland, VA (US); Benjamin G. Cameransi, JR., Georgetown, SC (US)

(73) Assignee: Lyotropic Therapeutics, Inc., Ashland, VA (US)

(21) Appl. No.: 14/138,279

(22) Filed: Dec. 23, 2013

Related U.S. Application Data
(63) Continuation of application No. 10/960,746, filed on Oct. 8, 2004, now abandoned.
Application No. 10/960,746 is a continuation-in-part of application No. 10/460,659, filed on Jun. 13, 2003, now abandoned.

(51) Int. Cl. A61K 31/445; A61K 9/14

(52) U.S. Cl. CPC A61K 31/445; A61K 9/141
USPC 424/490; 514/330; 424/489

(57) ABSTRACT

Drug formulations that provide sustained action and/or reduced dosage requirements are provided. In the formulations the drugs (particularly local anesthetics) are associated with reversed cubic phase and reversed hexagonal phase lyotropic liquid crystalline material.

### REFERENCE TO RELATED APPLICATIONS

This application is a continuation-in-part of U.S. patent application Ser. No. 10/460,659, filed Jun. 13, 2003, the complete contents of which are hereby incorporated by reference.

### BACKGROUND OF THE INVENTION

1. Field of the Invention

The present invention relates to drug formulations that provide sustained action and/or reduced dosage requirements. In particular, the invention provides therapeutic formulations in which the drugs, particularly local anesthetics, are associated with reversed cubic phase and reversed hexagonal phase lyotropic liquid crystalline material.

2. Background of the Invention

A number of methods have been used in the attempt to increase the duration of action of local anesthetics.

A method currently used in medical practice is the co-administration of vasoconstrictors such as epinephrine (adrenaline), phenylephrine, or norepinephrine, which increase the residence time of the drug at the site of administration, due to the induction of vasoconstriction with subsequent reduction of systemic uptake of the local anesthetic. While duration can be increased approximately two-fold for the short-acting local anesthetics, such as lidocaine, procaine, chloroprocaine, and prilocaine, this tends not to be the case with the longer-acting local anesthetics such as bupivacaine. Product literature from Astra-Zeneca on their currently marketed Marcaine® formulation of bupivacaine states: “The duration-prolonging effect of adrenaline is not as pronounced with bupivacaine as with the short-acting Local Anesthetics. Up to 50% prolongation, depending on mode of administration, can be seen.” Reproducible data consistently demonstrating local anesthetic nerve blocks lasting more than 10 hours from a single injection of local anesthetic at sub-toxic doses have only rarely been reported, even with co-administration of vasoconstrictive agents.

Epinephrine, along with other vasoactive agents, have their own toxicities and should be used with caution in certain patients. Cardiac arrhythmias may be produced in patients with heart disease or with the concomitant use of volatile anesthetic agents, such as halothane anesthesia. Hypertension may develop in patients with a pre-existing history of hypertension or with hyperthyroidism. In some cases, hypertension may be severe and actually trigger a hypertensive crisis. Epinephrine also has been demonstrated to be detrimental to the survival of delayed or expanded tissue flaps, since the new vessels present in these flaps appear to be exquisitely sensitive to the effects of epinephrine. Some workers prefer not to use local anesthetic solutions containing epinephrine on the helix of the ear or nasal alae. Other contraindications to the addition of epinephrine to local anesthetics include unstable angina, treatment with MAO inhibitors or tricyclic antidepressants, uteroplacental insufficiency, and peripheral nerve blocks in areas that may lack collateral blood flow (ear, nose, penis, digits).

Specifically with bupivacaine, tests in Sprague-Dawley rats have shown that the cardiotoxicity of bupivacaine (which is well-known to be dose-limiting, and has resulted in human deaths) is significantly increased by epinephrine, as well as by other vasoconstricting compounds. See J. R. Kambam, W. W. Kinney, F. Matsuda, W. Wright and D. A. Holaday (1990) Anesth. Analg. 70(5):543-5. While propanolol pretreatment can be used to protect against bupivacaine cardiotoxicity, this protective effect is largely abolished by the co-administration of epinephrine. See W. W. Kinney, J. R. Kambam and W. Wright (1991) Can. J. Anaesth. 38(4 Pt 1):533-6.

Additionally, several studies have demonstrated absence of prolongation by epinephrine in different nerve blocks. In a rat infraorbital nerve block model, little prolongation was seen with epinephrine in the case of bupivacaine, mepivacaine, prilocaine, or ropivacaine, and in some cases a significant reduction in duration was actually seen. See H. Renck and H. G. Hassan (1992) 36(5):387-92. Similarly, in a study in man of postoperative analgesia via a femoral catheter after total knee replacement, epinephrine did not increase the duration of analgesia from ropivacaine. See A. Weber, R. Fournier, E. Van Gessel, N. Riand and Z. Gamulin (2001) Anesth. Analg. 93(5):1327-31.

Significantly, the addition of epinephrine to tetracaine has recently been shown to greatly increase its neurotoxicity, apparently due to the induction of large glutamate concentrations, in the cerebrospinal fluid for an intrathecal administration; this represents a dangerous systemic toxicity. See S. Oka, M. Matsumoto, K. Ohtake, T. Kiyoshima, K. Nakakimura and T. Sakabe (2001) Anesth. Analg. 93(4):1050-7. Both sensory and motor dysfunction increased with epinephrine, as did vacuolation in the dorsal funiculus and chromatolytic damage of motor neurons.

These complications and others associated with epinephrine co-administration may well extend to any case where a vasoconstrictive substance is co-administered with the local anesthetic. Indeed, for the case of epidural administrations, Oka et al. (cited above) explicitly state that the increase in neuronal injury upon co-administration of epinephrine results directly from the vasoconstrictive effect. The results are likely to extend to local anesthetics in general.

From a practical perspective there are problems as well. Epinephrine is unstable at physiologic pH, so it is formulated at low pH, which results in significant pain on injection. Addition of bicarbonate to raise the pH can only be done at the time of injection, not significantly before, due to chemical degradation, making for a more complicated procedure. See Murakami et al. (1994) J. Dermatol. Surg. Oncol. 20(3):192.

Clonidine has been used to prolong the action of certain local anesthetics, but the prolongation is minor, less than about 50% and almost nothing in the case of the long-acting local anesthetics, yielding nerve block durations less than 8 hours in essentially all cases. Its use is thus limited mainly to cases where vasoconstrictive agents are contraindicated. Quite broadly, combinations of two active pharmaceutical ingredients (APIs) are disfavored when a single agent can achieve the same purpose.

Liposomal preparations of local anesthetics have demonstrated sustained action, but only at doses that vastly exceed the toxic dose of the LA. A representative example is given by Grant et al., in which a duration of about 24 hours was achieved in mice, but at a super-toxic dose—over 150 mg/Kg—that is 50 times the cardiotoxic dose. See G. J. Grant, B. Piskou and M. Bansinath (2003) Clin. Exp. Pharm. Physiol. 30:966. A nearly identical result is reported in U.S. Patent Application 2003/0185873 to Chasin et al., where Example 12 reports a dose of 150 mg/Kg. This dose also represents 150 times the recommended dose (1 mg/Kg), for human use. Similar results have been published from Grant et al. using neostigmine. Earlier results from Grant et al. claimed prolonged analgesia, but sensory blocks in mice using bupivacaine lasted only an average of just over 2 hours at doses that were in excess of 3 mg/Kg. See Grant et al. (1994) Regional Anesthesia 19(4):264. While those researchers pointed out that toxicity is reduced due to the encapsulation of the drug, the increase in dose to two orders of magnitude above the lethal dose would be a severe, and almost certainly insurmountable, impediment to approval by regulatory bodies and acceptance by the medical community. This is particularly true in the case of a liposomal preparation, because it is well known that liposomes are metastable, not stable, structures. Indeed, a dose 10 times, or even twice, the lethal dose would be severely problematic in any vehicle. (The term “vesicle” is alternatively used in place of “liposome”). Matrices based on lamellar phases, such as liposomes, can be of very low solubility, but generally rely on processes such as endocytosis or pinocytosis for interacting with cells, which are not only slow and inefficient but can result in an intact matrix trapped inside an endosome. Additionally, the solubilization of pharmaceutical actives of low water solubility in liposomes has not met with great success.

Sustained blood levels of bupivacaine were reported in a polycaprolactone microsphere formulation given either subcutaneously or intraperitoneally, but again, doses were far in excess of the cardiotoxic dose. Approximately 9.8 mg/Kg (2.46 mg per 250 gm rat) bupivacaine was administered, clearly a super-toxic dose, resulting in maximum plasma concentrations of about 240 ng/ml. See M. D. Blanco et al. (2003) Eur. J. Pharm. Biopharm. 55:229. Similar results were obtained using other polymer microspheres, namely bis-polcarboxyphenoxy-propane-sebacic acid anhydride (see D. B. Masters et al., 1993, Pharm. Res. 10:1527) and polylactide-glycolide (see P. LeCorre et al., 1994, Int. J. Pharm. 107:41).

A lipid emulsion containing bupivacaine has been reported that increases the duration of nerve block by approximately 30-40%, though curiously nerve blocks with that system lasted in duration only about 73 minutes (average of 3 animals). See Lazaro et al. (1999) Anesth. Analg. 89:121. These duration times were obtained under general anesthesia with phenobarbital and at a bupivacaine dose (approximately 3.2-3.6 mg/Kg) which are potentially cardiotoxic for bupivacaine. Furthermore, their formulation contains sodium oleate, which is presently not approved for injectable formulations nor does it belong to any class of compounds that contains a member that is approved for an injectable formulation.

Langerman et al. have reported a formulation of local anesthetics consisting of a solution of the drug in iophendylate. However, the intensity of nerve block—in other words, the efficacy—was reduced in the formulation, compared to the intensity using a simple aqueous solution at the same dose. See Langerman et al. (1992) Anesthesiology 77:475-81. It thus appears that obtaining an equally intense block would require an increase of dose, in comparison with the standard aqueous solution of local anesthetic currently approved and marketed. Also, aseptic arachnoiditis was reported after intrathecal administration of iophendylate. Indeed, arachnoiditis and severe irritation reactions, including death, have been frequently observed with iophendylate, which has been called more irritating and toxic than Lipiodol, a predecessor of iophendylate that was abandoned after severe adverse reactions. See E. Lindgren and T. Greitz (1995) Am. J. Euroradiology 16(2):351-60.

Dyhre et al. have published a study of lidocaine in a polar lipid formulation, in some cases together with dexamethasone which is an API known to prolong analgesia. Sciatic nerve blocks of increased duration were recorded, but only at doses of over 20 mg/Kg. This is far in excess of the maximum recommended dose of 7 mg/Kg. Indeed, doses of approximately 6 mg/Kg produced shorter durations of action that with the same dose of lidocaine hydrochloride in solution. Blood levels of lidocaine following perineural administration of the formulation were very high, at some time points two orders of magnitude higher than with the aqueous solution of lidocaine, which for many drugs would translate to increased risk of toxicity. See Dyhre et al., Acta Anesth. Scand. (2001) 45(5):583.

Furthermore, the polar lipid sunflower diglycerides used in the formulation of that study, and diglycerides in general, are not pharmaceutically-acceptable for intravenous injection (nor are monoglycerides).

### SUMMARY OF THE INVENTION

In the case of providing sustained-action drug delivery involving drugs of relatively low therapeutic index, it is crucial to note that dosage increase, which in most cases is tacitly assumed to be inevitable, is fraught with danger. This is illustrated particularly well by the case of local anesthetics, such as bupivacaine. In particular, the cardiotoxic dose is, for most local anesthetics, only modestly higher than the standard recommended dosage for nerve blocks. Specifically in the case of bupivacaine—one of the longest-acting local anesthetics and therefore one of the better drug choices for a prolonged-action formulation—the recommended dosage for nerve block is a maximum of 1.5 mg/Kg based on animal/human weight, while doses above 3 mg/Kg can be cardiotoxic or induce seizures. This rather low therapeutic index means that the usual methods of achieving sustained action, based on packaging larger amounts of drug in a formulation that releases it slowly—so as to maintain drug levels at or above the threshold level for efficacious action—inevitably require doses close to, or above, the toxic dose in a single administration. Because of the ever-present danger of inadvertent injection into a vein or artery, such an administration can be life-threatening, even in the case where the intended action of the vehicle is to release the drug slowly enough to reduce the risk of cardiotoxicity and seizures. A vehicle that requires, for example, more than 3 mg/Kg of bupivacaine, in order to achieve significant increase in duration of nerve block above the normal 2-5 hours, will introduce a risk of lethality that will not justify its routine use, either in the minds of regulatory bodies or in the medical community—regardless of what claims are made as to the safety of the vehicle. Any instability of such a vehicle, whether physical, chemical, shear-induced, temperature-induced, misapplication-induced, or shelf life-associated can in principle cause premature release of the drug, and if a substantial portion reaches the heart or brain, this would be risking serious adverse event, including death.

The basis of this invention is the surprising discovery that certain pharmaceutically-acceptable compositions are able to increase the duration of action of an active pharmaceutical ingredient (API) while avoiding the dose increase which is normally incumbent in sustained action formulations. The preferred method is to solubilize the drug in a reversed hexagonal phase or, most preferably, a reversed cubic phase liquid crystal material, and most preferably administer the material in the form of microparticles. Such a composition has the property that it increases the normal duration of action of that drug, preferably by more than about 50%, more preferably by 100%, and most preferably by 200%, or more, and in such a way that this increase in duration of action occurs with doses that are not super-toxic, and preferably sub-toxic, without introducing additional APIs or vasoconstrictive compounds. The preferred test is to evaluate the duration of nerve block, according to a procedure described in detail herein (see Example 2), of a formulation of bupivacaine in the composition; the duration, at a dose of 1 mg/Kg, should represent an increase, preferably of more than about 50%, of the normal 4 hour duration, in the case where no additional API is present. Most preferably this dose in such a formulation will yield a duration of action of more than about 10 hours. Additionally, administration of one-half the normal dose (which in the case of bupivacaine means 0.5 mg/Kg) should give at least the same efficacy and duration as 1 mg/Kg of the standard (single-agent) formulation (e.g. bupivacaine hydrochloride in aqueous solution). The surprising discovery at the core of this invention is that when these compositions are invoked, significantly prolonged duration of drug action can be achieved without increase of dose—indeed, even with a dramatically lower dose—which is particularly important in the case of drugs with low therapeutic index such as many local anesthetics. That long duration can be achieved without increasing drug dose, stands in sharp contrast with the normal state of affairs where significant increase in duration cannot be achieved without either increasing dose or adding another API and thus increasing potential risks, side effects, drug interactions, costs, and regulatory hurdles.

Preferred embodiments of this invention are compositions comprising a reversed cubic phase or reversed hexagonal phase liquid crystal, or a combination thereof, composed of pharmaceutically acceptable components. Such materials are in the class of lyotropic liquid crystals. They comprise a polar solvent (usually water), and a surfactant, of which poloxamers and polar lipids such as phospholipids are examples.

### DESCRIPTION OF THE PREFERRED EMBODIMENTS

The present invention provides methods that are useful for sustaining the action of APIs without increasing the administered dose. In many cases it is possible to decrease the dose of API and achieve the same or increased duration of action. In particular, the application of these methods to the delivery of local anesthetics yields results that confirm the effect of the compositions by achieving hitherto unachieved increases in duration at normal dose, and/or the same duration at significantly lower dose, and at the same time yields methods of administering local anesthetics that are of high potential utility in their own right. Of particular note is the disclosure herein of reversed liquid crystalline formulations of bupivacaine that yield nerve blocks of well over 16 hours in duration, where under identical conditions the currently marketed formulation yields 2-5 hours duration of nerve block. A closely related, liquid crystalline formulation of the anticancer drug paclitaxel yields excellent oral absorption leading to paclitaxel blood levels of extended duration.

Simple encapsulation and other simplistic slow-release methodologies have previously taught that dose increase is acceptable provided that slow release circumvents acute toxicity issues (not to mention that in many cases the tacit assumption has been made that dose increase is inevitable). However, as pointed out above, this is a naïve assumption, at least in the case of local anesthetics and other drugs of narrow therapeutic ratio, because an increase of dose calls for doses at or above threshold toxic doses too severe in consequence to be acceptable in a medical setting, particularly an elective, non-emergency, routine-use setting.

Preferred embodiments of the instant invention, which are able to achieve highly prolonged drug action without diminishment of efficacy or introduction of additional drugs, in a method that is pharmaceutically-acceptable even for intravenous injection, feature nanostructured liquid crystalline phases of the reversed type—namely reversed cubic and reversed hexagonal phases. These can be of very low solubility in water or show very slow dissolution kinetics, meaning that they maintain their integrity as vehicles, for at least some substantial period of time, upon entry into the body, thus avoiding drug precipitation or premature release, and show a great deal of promise in fields such as controlled-release drug delivery. In work motivated by the amphiphilic nature and porous nanostructures of these materials, which can lead to very advantageous interactions with biomembranes—much more intimate than in the case of liposomes and emulsion droplets—and by the high viscosities of these phases which can be an important aid in processing, a number of techniques have been developed for dispersing and encapsulating such materials.

### Definitions/Descriptions

In order to facilitate understanding of the present invention, the following definitions and descriptions of terms utilized herein are provided. Definitions of terms that appear, in turn, within these definitions (such as “surfactant”, “polar”, “apolar”, “amphiphile”, etc.) are provided in U.S. Pat. No. 6,638,621 to Anderson, the complete contents of which are herein incorporated by reference.

Pharmacologic agent: A material will be deemed a pharmacologic agent provided it is considered an Active Pharmaceutical Ingredient (API) by the pharmaceutical industry and by regulatory bodies (viz., the FDA in the United States), as opposed to an Inactive Ingredient (also known as an excipient). The term “drug” will be used interchangeably with “pharmacologic agent”, for brevity.

Efficacy: Efficacy is the specific ability or capacity of the pharmaceutical product to effect the result for which it is offered when used under the conditions recommended by the manufacturer. (This definition is taken verbatim from Title 9 of the United States Code of Federal Regulations). In the case of oral formulations of systemically-active drugs, drug efficacy is of course strongly affected by the degree of systemic absorption, as measured by the AUC (“Area Under the Curve”), an integration of blood levels over the time of duration of those blood levels.

Standard therapeutic dose; recommended dose: These terms, used interchangeably herein, refer to the dose that is, at the time of application of the pharmacologic agent, recommended for use in a given setting by authoritative sources in the pharmaceutical community, including the Physician's Desk Reference, package inserts of the drug product, and the Food and Drug Administration. The intention herein is that this refers to the dose when given in its standard vehicle—such as the aqueous solution of the hydrochloride form in the case of most local anesthetics—rather than in formulations as taught in this invention, for which we are using the standard formulation in the standard vehicle as a reference point.

Sub-toxic dose: An administered dose will be deemed “sub-toxic” in this disclosure if and only if it satisfies two criteria: 1) the amount of drug administered is less than or about equal to the highest generally accepted recommended dose for medical practice; and 2) the administered dose in the composition indicated does not introduce significant systemic toxicity in excess of that of the recommended dose in its standard vehicle. With respect to criterion 1, in the case of bupivacaine this criterion would require a dose less than about 2 mg/Kg; maximum recommended dosages of bupivacaine are provided in the Physician's Desk Reference (see, e.g., 55^{th} edition, page 601), and for a 70-Kg patient these doses translate to a maximum of about 2 mg/Kg.

Super-toxic dose: An administered dose will be deemed “super-toxic” in this disclosure if and only if it satisfies either of two criteria: 1) the amount of drug administered is greater than or about equal to the dose that is generally accepted to incur dangerous systemic toxicities; or 2) the administered dose in the composition indicated introduces dangerous systemic toxicities. (It will be noted that “super-toxic” is not synonymous with “not super-toxic”; rather there is a middle ground which is neither safe enough to satisfy the definition of “sub-toxic”, nor dangerous enough to fit the definition of “super-toxic”). With respect to criterion 1, in the case of bupivacaine this criteria for super-toxic translates to a dose in excess of 3 mg/Kg.

Baseline duration: The baseline duration of a pharmaceutical active means the average or typical duration of efficacious action for a basis dosage of that drug, which in most contexts herein will be understood to mean the published recommended dose. In the case of a local anesthetic this means the average duration of the analgesic or anesthetic action—herein defined to be sensory nerve block unless otherwise indicated—of that drug when given in its standard, aqueous, hydrochloride solution formulation according to the procedure that is standard medical practice (see the procedure described below). For bupivacaine, e.g., that baseline duration for a normal therapeutic dose of 1 mg/Kg is approximately 4 hours.

Single administration: A drug formulation will be deemed as given by a single administration if and only if the entire drug formulation is deposited in or on the body over a timescale that is at least an order of magnitude less than the baseline duration of that amount of drug when given in its standard vehicle, which in the case of a local anesthetic is an aqueous solution.

Increase in duration: The increase in duration of a drug given in a particular formulation is the ratio (expressed as a percentage) of the increment in time duration increase of efficacious action (in particular, for the case of a local anesthetic, this is the duration of nerve block, measured by the procedures described herein) of the drug in that formulation to the baseline duration of that same dose of same drug.

Relative duration: This is the increase in duration, plus 100%. That is, it is the ratio (expressed as a percentage) of the time duration of efficacious action of the drug in that formulation to the baseline duration of that same dose of same drug. A formulation with an increase in duration from, say, 4 hours to 6 hours would have an increase in duration of 50%, and a relative duration of 150%.

Relative dose: This is defined simply to be the ratio (expressed as a percentage) of the dose given in a particular formulation to the normal therapeutic dose (in particular, the dose referred to in the definition of baseline duration). For the case of bupivacaine, where the standard therapeutics dose is herein taken to be 1 mg/Kg, the relative dose of a formulation of interest is simply the dose divided by 1 mg/Kg, expressed as a percentage (that is, multiplied by 100%).

Amplification factor: This is defined to be the relative duration divided by the relative dose. As an example, in the case of the liposomal bupivacaine formulation of Grant et al. reviewed above, the relative dose was [150 mg/Kg]/[1 mg/Kg]×100%=15,000% and the relative duration was [24 hrs]/[4 hrs]×100%=600%, and so the amplification factor was 600%/15,000%=0.04.

Low therapeutic index; narrow therapeutic ratio: These terms will be used interchangeably. Narrow therapeutic ratio is defined in the regulations at 21 CFR 320.33(c). This subsection deals with criteria and evidence to assess actual or potential bioequivalence problems. Under Section 320.33(c) of Code of Federal Register 21, the US FDA defines a drug product as having a narrow therapeutic ratio as follows: there is less than a 2-fold difference in median lethal dose and median effective dose values, or there is less than 2-fold difference in the minimum toxic concentrations and minimum effective concentrations in the blood. For the purposes of this disclosure, the term will be interpreted more broadly, to indicate drugs for which the therapeutic window is sufficiently narrow that improvements in therapeutic index obtained by re-formulating the drug would be considered a significant advance in the field.

Pharmaceutically-acceptable: In the context of this invention, “pharmaceutically-acceptable” designates compounds or compositions in which each excipient is approved by the Food and Drug Administration, or a similar body in another country, for use in a pharmaceutical formulation, or belongs to a succinct class of compounds for which a Drug Master File or similar document is on file with a government regulatory agency, usually the FDA; this term is used herein in the context of a specific route of administration, e.g., “pharmaceutically-acceptable for intravenous injection”. The class of acceptable compounds also includes compounds that are major components of approved excipients, which are known to be of low toxicity taken internally; e.g., since peppermint oil is in a number of oral formulations, its major component menthol would have a similar status. A listing of approved excipients, each with the various routes of administration for which they are approved, was published by the Division of Drug Information Resources of the FDA in January, 1996 and entitled “Inactive Ingredient Guide”; this list is presently updated periodically on the FDA website. The existence of a Drug Master File at the FDA is evidence that a given excipient is acceptable for pharmaceutical use, at least for certain routes of administration. For injectable products, a listing of approved excipients was published in 1997. See Nema, Washkuhn and Brendel (1997) PDA J. of Pharm. Sci. & Technol. 51(4):166. It should be added that there are certain compounds, such as vitamins and amino acids, which are in injectable products, typically for parenteral nutrition, as “actives”, and are thus known to be safe upon injection, and such compounds are considered herein as pharmaceutically-acceptable as excipients as well, for injection. A particularly important example of a succinct class of compounds where a Drug Master File (DMF) is on file is the class of Pluronic (Poloxamer) surfactants, for which BASF has a DMF on file. In this case, although only a few members of this class have explicitly been used in injectable formulations, for the purposes of this invention, the homogeneity of the class, the presence of a DMF, and the existence of approved-for-injection formulations using several members of the class is sufficient to include each of the members of the class of Pluronics as pharmaceutically-acceptable for injectable products.

In the context of local anesthetics, the mistake is sometimes made that a local anesthetic formulation need only be pharmaceutically-acceptable for subcutaneous injection, or other local instillation. However, as pointed out elsewhere herein, the ever-present danger of inadvertent intravenous or intra-arterial injection of such a formulation leads directly to the requirement that the formulation be pharmaceutically-acceptable for intravenous injection. For a particulate vehicle, this also carries with it the important requirement that particle size be acceptable for i.v. injection, which usually means submicron, or preferably less than about 0.5 micron.

Excipient: compound and mixtures of compounds that are used in pharmaceutical formulations that are not the Active Pharmaceutical Ingredients themselves. The term “excipient” is synonymous with “inactive ingredient”.

Bilayer-associated (or membrane-associated): A compound or moiety is bilayer-associated if it partitions preferentially into a bilayer over an aqueous compartment. Thus, if a bilayer-rich material such as a reversed cubic phase material exists in equilibrium with excess water and is placed in contact with excess water, and a bilayer-associated compound or moiety is allowed to equilibrate between the two phases, then the overwhelming majority of the compound or moiety will be located in the bilayer-rich phase. The concentration of the compound or moiety in the bilayer-rich phase will be at least about 100 times, and preferably at least about 1,000 times, larger than in the water phase.

It is important to note that although the reversed hexagonal phases and reversed discrete or discontinuous cubic phases do not have a true bilayer as the fundamental structural unit, in the present disclosure we will nevertheless use the term “bilayer-associated” to describe components that partition into the lipid-rich (or surfactant-rich) microdomains irrespective of whether such domains are considered “monolayers” or “bilayers”. The term “bilayer-associated” is thus more directed to the partitioning of the compound in question than to the precise nature of the lipid (or surfactant) region.

Lyotropic liquid crystalline phases. Lyotropic liquid crystalline phases include the normal hexagonal, normal bicontinuous cubic, normal discrete cubic, lamellar, reversed hexagonal, reversed bicontinuous cubic, and reversed discrete cubic liquid crystalline phases, together with the less well-established normal and reversed intermediate liquid crystalline phases. These are discussed in detail in U.S. Pat. No. 6,638,621, the contents of which are hereby incorporated by reference.

The nanostructured liquid crystalline phases are characterized by domain structures, composed of domains of at least a first type and a second type (and in some cases three or even more types of domains) having the following properties:

a) the chemical moieties in the first type domains are incompatible with those in the second type domains (and in general, each pair of different domain types are mutually incompatible) such that they do not mix under the given conditions but rather remain as separate domains; (typically, the first type domains could be composed substantially of polar moieties such as water and lipid head groups, while the second type domains could be composed substantially of apolar moieties such as hydrocarbon chains, fused ring systems, polypropyleneoxide chains, polysiloxane chains, etc.);

b) the atomic ordering within each domain is liquid-like rather than solid-like, lacking lattice-ordering of the atoms; (this would be evidenced by an absence of sharp Bragg peak reflections in wide-angle x-ray diffraction);

c) the smallest dimension (e.g., thickness in the case of layers, diameter in the case of cylinders or spheres) of substantially all domains is in the range of nanometers (viz., from about 1 to about 100 nm); and
d) the organization of the domains conforms to a lattice, which may be one-, two-, or three-dimensional, and which has a lattice parameter (or unit cell size) in the nanometer range (viz., from about 5 to about 200 nm); the organization of domains thus conforms to one of the 230 space groups tabulated in the International Tables of Crystallography, and would be evidenced in a well-designed small-angle x-ray scattering (SAXS) measurement by the presence of sharp Bragg reflections with d-spacings of the lowest order reflections being in the range of 3-200 nm.

Reversed hexagonal phase: The reversed hexagonal phase is characterized by:
1. Small-angle x-ray shows peaks indexing as 1:✓3:2:✓7:3 . . . in general, ✓(h²+hk−k²), where h and k are integers—the Miller indices of the two-dimensional symmetry group,
2. To the unaided eye, the phase generally transparent when fully equilibrated, and thus, e.g., often considerably clearer than any nearby lamellar phase.

3. In the polarizing optical microscope, the phase is birefringent, and the wellknown textures have been well described by Rosevear and by Winsor (e.g., Winsor (1968) Chem. Rev., p. 1). The most distinctive of these is the “fan-like” texture. This texture appears to be made up of patches of birefringence, where within a given patch fine striations fan out giving an appearance reminiscent of an oriental fan. Fan directions in adjacent patches are randomly oriented with respect to each other. A key difference distinguishing between lamellar and hexagonal patterns is that the striations in the hexagonal phase do not, upon close examination at high magnification, prove to be composed of finer striations running perpendicular to the direction of the larger striation, as they do in the lamellar phase.
4. Viscosity is generally quite high,; the zero-shear limiting viscosity is in the range of millions or even billions of centipoise.
5. The self-diffusion coefficient of the water is slow compared to that in the lamellar phase, at least a factor of two lower; that of the surfactant is comparable to that in the reversed cubic and lamellar phases.
6. The ²H NMR bandshape using deuterated surfactant shows a splitting, which is one-half the splitting observed for the lamellar phase.
7. In terms of phase behavior, the reversed hexagonal phase generally occurs at high surfactant concentrations in double-tailed surfactant/water systems, often extending to, or close to, 100% surfactant. Usually the reversed hexagonal phase region is adjacent to the lamellar phase region that occurs at lower surfactant concentration, although bicontinuous reversed cubic phases often occur in between. The reversed hexagonal phase does appear, somewhat surprisingly, in a number of binary systems with single-tailed surfactants, such as those of many monoglycerides, and a number of nonionic PEG-based surfactants with low HLB.

Reversed cubic phase: The reversed cubic phase is characterized by:
1. Small-angle x-ray shows peaks indexing to a three-dimensional space group with a cubic aspect. The most commonly encountered space groups, along with their indexings are:
Ia3d (#230), with indexing ✓6:✓8:✓14:4 . . . Pn3m (#224), with indexing ✓2:✓3:2:✓6:✓8: and Im3m
(#229), with indexing ✓2:✓4:✓6:✓8:✓10 . . . . The cubic space groups #212 (derived from that of space group #230 by a symmetry break) and #223 (corresponding to closed micelles arranged on a cubic lattice) have also been observed.
   2. To the unaided eye, the phase is generally transparent when fully equilibrated, and thus often considerably clearer than any nearby lamellar phase.
   3. In the polarizing optical microscope, the phase is non-birefringent, and therefore there are no optical textures.
   4. Viscosity is very high, much more viscous than the lamellar phase. Most reversed cubic phase have zero-shear viscosities in the billions of centipoise.
   5. No splitting is observed in the NMR bandshape, only a single peak, corresponding to isotropic motion.

6. In terms of phase behavior, the reversed bicontinuous cubic phase is found either between the lamellar phase and the reversed hexagonal phase, or to lower water content than the reversed hexagonal phase. A good rule is that if the cubic phase lies to higher water concentrations than the lamellar phase, then it is normal, whereas if it lies to higher surfactant concentrations than the lamellar then it is reversed (a notable exception being the case of the reversed cubic phase in long-chain unsaturated monoglycerides). The reversed cubic phase generally occurs at high surfactant concentrations in double-tailed surfactant/water systems, although this is often complicated by the fact that the reversed cubic phase may only be found in the presence of added hydrophobe (‘oil’) or amphiphile. The reversed bicontinuous cubic phase does appear in a number of binary systems with single-tailed surfactants, such as those of many long-chain monoglycerides (include glycerol monooleate), and a number of nonionic PEG-based surfactants with low HLB.

Dehydrated variants. A dehydrated variant of a reversed liquid crystal is a composition that yields a reversed liquid crystalline phase upon contact with water (or more rarely, other polar solvent)—whether or not this dehydrated composition itself is a reversed liquid crystalline phase.

### Methods

In the practice of this invention, the composition should preferably be such that it accomplishes solubilization of the drug at sufficiently high concentrations that vehicle volumes are kept reasonable, from the point of view of both a volume of administration and a toxicity. (That is, as the drug concentration in the vehicle goes down, the amount of each excipient required to administer a given dose goes up, eventually reaching levels where low toxicity is compromised). In the case of local anesthetics with amino groups, it is preferred that the local anesthetic be solubilized substantially in its non-protonated (or “free base”) form. This increases the partition coefficient of the drug into the hydrophobic domains of the vehicle. Methodology and compositions for solubilizing local anesthetics as well as a wide range of other drugs in reversed liquid crystalline materials are discussed at length in International PCT application ______ entitled Drug-Delivery Vehicles Based on Reversed Liquid Crystalline Phase Materials, filed on Oct. 8, 2004, as well as in U.S. Pat. Nos. 6,482,517 and 6,638,621 both to Anderson, the contents of which are hereby incorporated by reference.

The physical form of these reversed liquid crystalline phases can take a number of useful forms. Bulk liquid crystal can be applied in a number of ways, including: topically, as a cream or ointment; buccally or sublingually; by injection such as subcutaneous or intramuscular; and orally, as for example inside a gel capsule. Microparticle formulations—suspensions or dispersions of particles—are preferred, particularly since they can, if prepared properly as exemplified in the Examples herein, be injected intravenously (which can be of tremendous importance in the case of local anesthetics and other injectable actives that can be toxic upon inadvertent intravenous or intra-arterial administration); microparticle formulations are especially versatile in that they can be given subcutaneously, intramuscularly, intrathecally, intraperitoneally, intrapleurally, intralymphatically, intralesionally, intradermally, subdermally, intraocularly, epidurally, etc., or given orally, intranasally, by inhalation, or rectally, in addition to intravenously under conditions discussed herein.

Particles of reversed liquid crystalline material. Two fundamental types of reversed liquid crystal-based microparticle formulations are coated (or “shelled”), and uncoated. Coated particles and methods of making them are described in detail in U.S. Pat. Nos. 6,482,517 and 6,638,621 both to Anderson, the contents of which are hereby incorporated by reference. Coated particles featured in Examples 2, 6 and 7 below are shown to yield very prolonged drug action without increase of, or with decrease of, dose.

An uncoated particle of reversed cubic (or hexagonal) phase is a particle in which the outermost material phase of the particle is a reversed cubic (or hexagonal) phase, so that there is no other phase present exterior to and in contact with this outermost material phase except for a single liquid (usually aqueous) phase in which the particles are dispersed (known as the continuous phase, or exterior phase), and wherein the material of this reversed cubic [hexagonal] phase is a single, contiguous and isolated mass of material thus defining a single particle. In this definition “isolated” means substantially not in contact with other such particles except for the normal particle-particle collisions in the course of Brownian motion.

The process for making such uncoated particles can be described as follows, and is illustrated in more detail in Examples 3, 4, and 5 below. Along with the selection of liquid crystal composition, one or more appropriate ionically-charged, bilayer-associated components is/are selected based on such properties as partition coefficient (generally high is best, preferably greater than about 1,000), low toxicity, favorable regulatory status (dependent on the route of administration), and solubility and compatibility with the other components of the formulation. A selection of such components is given herein. After adding the charged component to either the liquid crystal or to the exterior (usually aqueous) phase, the liquid crystal is homogenized into the exterior phase by homogenization, microfluidization, and/or filtration. Once the zeta potential of a collection of these reversed liquid crystalline phase particles equals or exceeds about 25 millivolts in magnitude (that is, more positive than 25 mV or more negative than −25 mV), or preferably greater than about 30 mV in magnitude (or more negative than −30 mV), then no other mechanism is required for stabilization of the dispersion against flocculation.

For formulations intended for administration by injection or other non-oral routes, especially preferred anionic moieties for stabilizing particle dispersions are: docusate, dodecylsulfate, deoxycholic acid (and related cholates, such as glycocholate), tocopherol succinate, stearic acid and other 18-carbon fatty acids including oleic, linoleic, and linolenic acids, gentisic acid, hydrophobic amino acids including tryptophan, tyrosine, leucine, isoleucine, aspartic acid, cystine, and their N-methylated derivatives, particularly N-acetyltryptophan, as well as phosphatidylserine, phosphatidylinositol, phosphatidylglycerol (particularly dimyristoyl phosphatidylglycerol), and other anionic and acidic phospholipids. The person with skill in the art will recognize docusate as the anionic moiety of the surfactant docusate sodium (also known as Aerosol OT), and dodecylsulfate as the anionic moiety of the surfactant sodium dodecylsulfate, or SDS. Preferred cationic stabilizers are: benzalkonium chloride, myristyl-gamma-picolinium chloride, and to a lesser extent tocopheryl dimethylaminoacetate hydrochloride, Cytofectin gs, 1,2-dioleoyl-sn-glycero-3-trimethylammonium-propane, cholesterol linked to lysinamide or ornithinamide, dimethyldioctadecyl ammonium bromide, 1,2-dioleoyl-sn-3-ethylphosphocholine and other double-chained lipids with a cationic charge carried by a phosphorus or arsenic atom, trimethyl aminoethane carbamoyl cholesterol iodide, O,O′-ditetradecanoyl-N-(alpha-trimethyl ammonioacetyl) diethanolamine chloride (DC-6-14), N-[(1-(2,3-dioleyloxy)propyl)]-N—N—N-trimethylammonium chloride, N-methyl-4-(dioleyl)methylpyridinium chloride (“saint-2”), lipidic glycosides with amino alkyl pendent groups, 1,2-dimyristyloxypropyl-3-dimethylhydroxyethyl ammonium bromide, bis[2-(11-phenoxyundecanoate)ethyl]-dimethylammonium bromide, N-hexadecyl-N-10-[O-(4-acetoxy)-phenylundecanoate]ethyl-dimethylammonium bromide, 3-beta-[N—(N′,N′-dimethylaminoethane)-carbamoyl. Surface-active polypeptides and proteins, such as casein and albumin, may also be used as charged stabilizers, although careful attention must be paid to the pH, which will have an effect on the charge of the molecule.

Dehydrated materials. It can be advantageous in certain circumstances to use a composition that yields a reversed liquid crystalline phase upon contact with water (or less preferably, other polar solvent)—whether or not this dehydrated composition itself is a reversed liquid crystalline phase. In particular, this contact with water or a water-containing mixture could be either during a reconstitution step, or more preferably, during the application of the particle, most preferably after the coating releases, and the de-coated particle contacts an aqueous solution such as blood, extracellular fluid, intracellular fluid, mucous, intestinal fluid, etc. There are several reasons why this may be advantageous: to protect hydrolytically unstable actives or excipients; for maintenance of sterility; to limit premature release of water-soluble actives; and as a natural result of a production process such as spray-drying or freeze-drying that can induce dehydration. Removal of most, or all, of the water from a reversed liquid crystalline phase will often yield another nanostructured liquid or liquid crystalline phase, but can sometimes yield a structureless solution, precipitate, or a mixture of these with one or more nanostructured liquid or liquid crystalline phases. In any case, for many applications, it is the hydrated form that is important in the application of the particles, and thus if this hydrated form is a reversed liquid crystalline phase, then the composition of matter falls within the scope of the current invention. There are three general ways in which such a particle can be produced. One is to use a process where a matrix or, in this case dehydrated matrix, is dispersed in a non-aqueous solution or melt that is, or contains, a precursor of the coating material; upon cooling or otherwise converting this precursor to the coating, the dehydrated matrix would then be the encapsulated entity. A second general method is to apply a drying process, such as freeze-drying, electrospinning, or preferably spray-drying, to a water-containing dispersion or preparation of the particles in which a coating material (or a precursor thereof) or dispersant/disintegrant has been dissolved or very finely dispersed. And a third general method is to dissolve or disperse all the components of the coating or dispersant and of the matrix, either including or excluding the water, in a volatile solvent and applying a drying process, again preferably spray-drying. Several of these methods can avoid the use of water completely, which would be important in the case of actives (or special excipients) that should not contact water even during production.

Especially preferred surfactants. The cubic and hexagonal phases described herein have a number of unique properties, and significant advantages over cubic and hexagonal phases that have been described in the literature, particularly as relate to their potential application in drug-delivery, and in the closely related fields of cosmeceutics and nutriceuticals. The problems and limitations associated with many of the lipids used in the prior art in making reversed cubic and reversed hexagonal phases for solubilizing actives, including toxicity and regulatory problems, limited ability to incorporate hydrophobes that are useful for solubilizing actives (in the case of monoglycerides), expense (in the case of galactolipids), and inappropriate phase behavior, are substantially eliminated in the compositions reported in this disclosure. The low-solubility poloxamers, with identification numbers ending with a “3” or more preferably a “2” (such as poloxamer 402, also known as Pluronic L122), form reversed liquid crystalline phases that include substantial levels of hydrophobes (“oils”, such as essential oils or components thereof, tocopherols, etc.), often over 20% by volume, and are as a result excellent matrices for solubilizing drugs in the current invention. Unsaturated phosphatidylcholines (such as PC-rich preparations from plant lecithins) similarly take up high levels of oils, as discussed in U.S. Pat. No. 6,638,621 to Anderson, the complete contents of which are herein incorporated by reference. It should be noted that, as illustrated in many of the Examples reported herein, the local anesthetic bupivacaine is solubilized in its low-solubility, free base form in a liquid crystal containing a solubilizing oil. This liquid crystal formulation with the free base form so solubilized provides an environment into which the bupivacaine partitions strongly, since the value of K_{ow} is approximately 1500. The inventor has found the following pharmaceutically-acceptable surfactants to be particularly useful in forming insoluble reversed cubic and hexagonal phases: phosphatidylcholine, Arlatone G and other low-HLB polyoxyethylated castor oil derivatives, Tween 85, glycerol monooleate and other long-chain unsaturated monoglycerides (for oral, topical/transdermal, and buccal only), sorbitan monooleate, zinc and calcium docusate, and as stated above, Pluronics with less than or equal to about 30% PEO groups by weight, especially Pluronic L122 and to a lesser extent L101 and P123.

Application to nerve block. Introducing (or Placing) local anesthetics at or in proximity to neural tissue results in anesthesia or analgesia and is broadly referred to as regional anesthesia. Specific techniques have evolved to establish surgical anesthesia, post-operative analgesia, as well as various acute and chronic pain management therapies. These techniques continue to evolve as advancements are made in pharmaceutical agents, medical devices, and the understanding of physiology and cellular function. Certain of these specific techniques are occasionally referred to as “nerve block”, “nerve root block”, “neural block”, “neuraxial block”, “intrathecal block”, “subarachnoid block” “epidural block”, “ganglion block”, “plexus block”, “field block”, “incisional block”, “infiltration block” among others. The current invention is of potential importance in all of these blocks.

The class of pharmacologic agents widely referred to as local anesthetics all posses the ability to reversibly block the dynamic conduction of nerve impulse along a nerve pathway. The site of activity is widely believed to be at the level of the axonal membrane. Furthermore it is believed that local anesthetics affect the axonal membrane by altering or preventing the flux of Na⁺ (sodium ion). This alteration increases the threshold for electrical excitation within the nerve which decreases or eliminates conduction impulse propagation, reducing the “rate of rise” of the action potential. This interruption, when effected over a distance, serves to block the conduction of nerve electrical impulses.

The term “differential blockade” is used to describe the various effects observed when various local anesthetics are used to establish regional anesthesia upon differing types of nerve fibers. The use of each local anesthetic agent will yield varying characteristic results based in large part on the agent's inherent hydrophobic or hydrophilic properties. Equally as important to the drug selected for use is the type of nerve fiber to be blocked of activity. Nerve fibers are typically classified by diameter and the presence or absence of myelin sheathing. A widely recognized Classification of Nerve Fibers has been established and is comprised of three major classes. The “A fibers” are myelinated somatic nerve fibers, the “B fibers” are myelinated pre-ganglionic autonomic nerve fibers, and the “C fibers” are non-myelinated post-ganglionic sympathetic nerve fibers.

Certain other factors can affect the quality or characteristic of a specific type of nerve block. Historically, rapidity of onset and duration of conduction blockade can be manipulated by increasing the total dose of the local anesthetic as well as the volume of delivery. The addition of epinephrine, norepinephrine, and phenylephrine can increase the duration of blockade due in large part their vasoconstrictive effects that reduce the absorption of the local anesthetic away from the nerve fiber. The proximity of the nerve fiber and other anatomic structures located near the injection site can affect the onset and duration of the block. Any number of independent factors including, but not limited to pH, bicarbonation, carbonation, temperature, baricity, the hormone progesterone, can effect the characteristic onset, quality, and latency of various nerve conduction techniques.

In man, blockade of the sciatic nerve may be performed to yield anesthesia distal to the lower extremity distal to the knee and to the foot. There are a number of prescribed regional anesthesia techniques that result in successful conduction block, primarily by using either the peripheral or classic approach. Either series of techniques may be performed either with the aid of a peripheral nerve stimulator or without, by eliciting parasthesias combined with the knowledge of anatomical and surface landmarks.

Use of a nerve stimulator generally facilitates the precise delivery of a local anesthetic agent in direct proximity to and even within the nerve and nerve sheath. This is accomplished by applying a small and adjustable amount of electric current to an insulating block searching needle to cause depolarization of the nerve once the non-insulated needle tip is advanced to a location near or against the nerve. This technique aids the trained practitioner in the identification and isolation of the nerve(s) intended to be blocked.

In the example of a sciatic nerve block to be performed in the lateral Sim's position, the leg intended to be blocked would be flexed at the knee and the uppermost extremity, resting on the dependent lower extremity. By palpation, one would identify the greater trochanter and ischial tuberosity in order to identify the anatomic notch between the two key landmarks. The sciatic nerve lies nearly midpoint within this notch. The corresponding surface of the skin above this point will be anesthetized by injecting a small amount of a local anesthetic by raising a skin wheal. The negative lead of the nerve stimulator is then attached in proximity to the needle hub, and the tip of the block needle is then advanced into the sciatic notch. As the needle is advanced, both dorsiflexion and plantar flexion of the foot will be observed once proximity of the needle tip to the nerve has been established. Confirmation of needle placement may be made by either decreasing the electrical stimulation to less than 0.2 milliamps or by injected 1 to 2 milliliters of local anesthetic, which will abolish sufficient electrical stimulation and cause a diminishment and eventual loss of the motor movement. The sciatic block may then be completed by delivering an appropriate amount of local anesthetic solution to the sedated adult or anesthetized child.

The following are examples of nerve blocks that may offer an improved level of comfort with a longer lasting local anesthetic as provided in this invention.

HEAD & NECK

| | | |
|---|---|---|
| Tonsils & Adenoids | Palatine fossa block | *** |
| Lymph node biopsy, neck | superficial cervical plexus block | ** |
| Carotid endarterectomy | superficial/deep cervical plexus b. | ** |
| General post-op pain control | superficial and deep incisional injection | * |
| RSD/Causalgia/Raynauds | stellate ganglion block | *** |

UPPER EXTREMITY

| | | |
|---|---|---|
| Shoulder arthroscopy, diagnostic | brachial plexus, interscalene approach | * |
| Open or scope, rotator cuff repair | b.p., interscalene approach | *** |
| Arthroplasty, | b.p., interscalene approach | *** |
| ORIF humeral fx | b.p, interscalene approach | * |
| Arm ORIF olecranon fx (elbow) | brachial plexus, axillary approach | ** |
| Olecranon bursa | brachial plexus, axiallary/infraclavicular | ** |
| Fore Arm ORIF radius, ulna | brachial plexus, axillary/infraclavicular | *** |
| Dialysis shunt insertion | ″ | * |
| Wrist, hand, digit | selective site block(s) | * to ** |

THORAX & ABDOMINAL WALL

| | | |
|---|---|---|
| Chest, thoracotomy (open chest) | paravertebral block (T1-T6 0r T8) | *** (#) |
| Pain control, fx rib(s) | paravertebral/intercostals | *** (#) |
| Shingles (dermatomal) | intercostal nerve block(s) | * |
| Mastectomy/axillary lymph node | paravertebral (T1-T6) | *** |
| Breast reconstruction without abdominal tran/flap | ″ | *** (#) |
| Breast reconstruction with abdominal tran/flap | ″ | none |
| Inguinal hernia patch & plug, open, w/mesh | | * |

PELVIS, PERINIUM, UROGENTIAL

| | |
|---|---|
| Various site specific blocks | * |

LOWER EXTREMITY

| | | |
|---|---|---|
| Knee arthroscopy, diagnostic | lumbar plexus, femoral n block | * |
| Knee scope w/repair ligaments | ″ | ** |
| Total knee arthroplasty | ″ | *** (#) |
| ORIF patella | ″ | ** |
| Total hip arthroplasty | ″ | * |
| Amputation, above/below knee | sciatic nerve block | ** |
| Distal leg ORIF, tibia | ″ | ** |
| Foot, ankle, tendons | popliteal nerve, ankle block | * to *** |

Legend
* some improvement offered over 4 to 6 hr marcaine ™ block
** good improvement likely compared to marcaine ™ block
*** significant improvement offered over single shot marcaine ™ block
(#) thoracic or lumbar level epidural indwelling catheter offers significant advantages over single shot Marcaine ™ or the current invention, though suffers from certain issues.

Application to other actives. Pharmaceutical compounds that are well-suited for incorporation as actives in the instant invention, most preferably into the reversed cubic phase liquid crystalline materials of the preferred embodiments, and could potentially reap benefit from the methods of the present invention, include propofol, alphaxalone, alphadolone, eltanolone, propanidid, ketamine, pregnanolone, etomidate, and other general anesthetics; dexamethasone, clonidine, loperamide, serotonin antagonists like ondansatron, especially in conjunction with certain local anesthetics; amphotericin B; coenzme Q10; steroids and steroidal anti-inflammatory agents; epoietin; mitoxanthrone; dacarbazine; nonsteroidal anti-inflammatories (e.g., salicylates, para-aminophenol derivatives (e.g., acetaminophen); calcitonin; sucralfate; danazol and other steroids; megace; L-dopa; ketamine; acyclovir and other antivirals; anakinra; flavanoids (nutriceuticals); fenomates; pentafuside; proprionic acid derivatives (e.g., naproxen, ibuprofen, etc.); analgesics; antipyretics; neuromuscular blocking agents such as rocuronium, vecuronium, and pancuronium; antihypertensives, such as sulfinalol, oxyprenolol, hydrochlorothiazide, captopril, felodipine, guanazodine, cadralazine, tolonidine, pentamethonium bromide, bunazosin, ambuside, methyldopa, etc.; antitussives, such as mutamirate, etc.; sedatives (e.g., benzodiazepines such as diazepam); hypnotics (e.g., intravenous anesthetics and barbiturates); opiates; cannabinoids and proteins (e.g., insulin and erythropoietin). The local anesthetics are of course especially preferred within the context of this invention, and include bupivacaine, lidocaine (which has a low therapeutic index, in spite of its use against ventricular arrhythmias), procaine, tetracaine, mepivacaine, etidocaine, oxybuprocaine, cocaine, benzocaine, pramixinine, prilocaine, proparacaine, ropivicaine, levobupivacaine, amylocaine, dibucaine, diperodon, hexylcaine, leucinocaine, meprylcaine, chloroprocaine, dibucaine, oxybutacaine, propanocaine, propipocaine, pseudococaine, butacaine, QX-314, and related local anesthetics; dental anesthetics such as chlorobutanol, eugenol, and clove oil; and a 1:1 by weight eutectic mixture of lidocaine and prilocaine. Antineoplastic drugs generally have narrow therapeutic ratios and can benefit especially from this invention; these include SN-38 and related camptothecins such as irinotecan; paclitaxel and related taxanes; gemcitabine; colchicine; doxorubicin, idarubicin, daumorubicin and related rubicins; illudins and the related ptaquilosin; filgrastime; vincristine and vinblastine; perindopril; epothilones; photofrin and other PDT agents; cyclophosphamide; 13-cis-retinoic acid; clotrimazole (for oral thrush); cisplatin, carboplatin, and other platinum-based drugs. In addition, other pharmaceutical compounds listed in U.S. Pat. Nos. 6,638,537 and 6,638,621, the complete contents of which are herein incorporated by reference, are suitable for incorporation into the invention described herein, and preferably into the reversed liquid crystalline phases of the preferred embodiments; one of the Examples below details an application of the invention to an antineoplastic agent, namely a taxane, paclitaxel. In addition, other drugs and neutriceuticals which are of low therapeutic index and are especially preferred for the current invention include warfarin and other anticoagulants, cyclosporin and other immunosupressives including basiliximab, antifungal agents, digoxin, phenytoin, theophylline, aminophylline, lithium, aminoglycoside antibiotics, insulin, dimercaprol, mercaptopurine, fluoroquinolones, antiepileptic drugs, oral contraceptives, phenylpropanolamine, trypanocidal compounds, vitamins A and D, quinidine, miltefosine, terfenadine, hormones, cisapride, 3-hydroxy-3-methylglutaryl coenzyme A reductase inhibitors, potent narcotic analgesics such as fentanyl and buprenorphine, many psychotropic drugs such as butaclamol, many MAO inhibitors, and tricyclic depressants, and to some extent the barbiturates. Broadly speaking, any drug for which chiral separations have been carried out in order to remove the enantiomer of lower therapeutic index is likely to be a preferred candidate for this invention.

It should also be pointed out that the current invention could play a role in facilitating the use of certain pharmaceutical actives which have gone out of favor due to drug abuse problems, such as cocaine. By changing the physical form and the pharmacokinetics of the drug through the use of this invention, pharmaceutical efficacy could be preserved, or improved, while discouraging or precluding the possibility of abuse.

Routes of Administration. The compositions of the present invention may be administered by any of a variety of means that are well known to those of skill in the art. These means include but are not limited to oral (e.g. via pills, tablets, lozenges, capsules, troches, syrups and suspensions, and the like) and non-oral routes (e.g. parenterally, intravenously, intraocularly, transdermally, via inhalation, and the like). The compositions of the present invention are particularly suited for internal (i.e. non-topical) administration. The present invention is especially useful in applications where a difficultly soluble pharmaceutical active is to be delivered internally (i.e. non-topical), including orally and parenterally, wherein said active is to be miscible with a water continuous medium such as serum, urine, blood, mucus, saliva, extracellular fluid, etc. In particular, an important useful aspect of many of the structured fluids of focus herein is that they lend themselves to formulation as water continuous vehicles, typically of low viscosity.

### EXAMPLES

The following examples illustrate the present invention but are not to be construed as limiting the invention.

### Example 1

The surfactant Pluronic 123, combined with water and a number of non-paraffinic hydrophobes, were found to form reversed cubic phases at specific compositions. The compositions found included the following reversed cubic phase compositions:

Pluronic 123 (47.8%)/orange oil (26.1%)/water (26.1%);

Pluronic 123 (45.7%)/isoeugenol (21.7)/water (32.6%); and

Pluronic 123 (47.8%)/lemon oil (26.1%)/water (26.1%).

Furthermore, these cubic phases are capable of solubilizing drugs of low solubility. Free base bupivacaine (solubility in water less than 0.1% by wt) was made by dissolving 1.00 g of bupivacaine hydrochloride in 24 mL water. An equimolar amount of 1N NaOH was added to precipitate free base bupivacaine, which was then freeze-dried. In a glass test tube, 0.280 g free base bupivacaine, 0.685 g water, and 0.679 g linalool were combined and sonicated to break up bupivacaine particles. Then 0.746 g of the surfactant Pluronic P123 (poloxamer 403) was added. The sample was stirred and heated to dissolve the crystalline drug. The sample was centrifuged for fifteen minutes. The sample had formed a highly viscous, clear phase that was optically isotropic in polarizing microscopy.

A second sample was also prepared using the same liquid crystal, then formulating it into microparticles coated with zinc tryptophanate. These bupivacaine-loaded microparticles are suitable for subcutaneous injection, as a slow-release formulation of the local anesthetic with the purpose of prolonging the drug's action and lowering its toxicity profile.

These two samples were then examined by small-angle X-ray scattering. The data were collected on a small angle x-ray line with copper radiation, Frank mirrors, an evacuated flight path and sample chamber, a Bruker multi-wire area detector, and a sample-to-detector distance of 58 cm (d-spacing range of 172 to 15 angstroms). Since the highest d-spacing observed on this sample was close to the limit of detection with this camera, it was also run on a 6-meter 2D small angle x-ray line with copper radiation, Osmic multi-layer optics, pinhole collimation, an evacuated flight path, helium-filled sample chamber and a Bruker multi-wire area detector and a sample-to-detector distance of 328 cm. At 328 cm the detector has a range of 90 to 700 Angstroms. The first material was loaded into a 1.5 mm i.d. x-ray capillary from Charles Supper Corp. The sample was run at 18 C. The two-dimensional images from the 58 cm distance were integrated with a step size of 0.02 degrees two-theta. Data from the 6-meter line were integrated with a step size of 0.002 degrees two-theta and those plots were overlaid with the runs at the shorter distance, and excellent agreement was obtained between the peak positions recorded with the two cameras.

The x-ray peak analysis software program JADE, by Materials Data Analysis, Inc., was used to analyze the resulting data for the presence and position of peaks. Within that program, the “centroid fit” option was applied.

The SAXS data show Bragg peaks determined by JADE at positions 154.6, 80.6, 61.6, and 46.3 Angstroms. These peaks index to a cubic phase structure of the commonly-observed cubic phase space group of Pn3m (see Pelle Strom and D. M. Anderson, Langmuir, 1992, vol. 8, p. 691 for a detailed discussion of the most commonly observed cubic phase structures and their SAXs patterns). These four peaks in fact index as the (110), (211), (222) and (420) peaks of this space group (#229), with a lattice parameter of 210 Angstroms. The second sample exhibited one peak, at 104.6 Angstroms, which appears to index as the (200) peak of the same lattice. The second sample also showed three peaks with d-spacings less than 25 Angstroms, which were clearly due to the crystalline zinc tryptophanate shell.

Isoeugenol is a major component of ylang-ylang oil and other essential oils, and has been the focus of a number of toxicity studies demonstrating its low toxicity. Linalool is a major component of coriander oil as well as other essential oils such as cinnamon, and orange oils, and is considered non-paraffinic according to the definition given above because the maximum length of saturated hydrocarbon chain is only 5; the non-paraffinic nature of this compound is underscored by the presence of not only unsaturated bonds but also branching, tertiary carbons, and a hydroxyl group. Linalool has also been the subject of intensive toxicity studies that nearly universally show low toxicity and mutagenicity, and in particular the LD50 for subcutaneous injection in mice was reported to be 1,470 mg/Kg. See NIEHS report prepared by Technical Resources International, Inc. under contract No. NO2-CB-50511, June 1997, revised September 1997.

The Pluronics (also called Poloxamers) are a rich class of surfactants that include variants covering a wide range of molecular weights and HLBs (hydrophilic-hydrophobic balance). Those with low HLBs are of low water solubility, especially if they are of high MW, and P123 is an example of such a surfactant that nonetheless has a large enough PEG group to form self-association structures under a wide range of conditions. Furthermore its relatively high MW also encourages the formation of liquid crystalline (as opposed to liquid) phases, which is very favorable in the present context. Pluronics are also known to interact strongly with biomembranes so as to enhance cellular absorption of drugs, and may in fact inhibit certain efflux proteins, such as P-glycoprotein and other MDR proteins that are responsible for multidrug resistance. Phosphatidylcholine, for example, has not been shown, or to this author's knowledge even speculated, as performing the latter function in drug-delivery. Pluronics as a class are the subject of a Drug Master File with the FDA, and a number are listed explicitly on the 1996 Inactive Ingredient list as being approved for injectable formulations, indicating their low toxicity.

### Example 2

The cubic phase of Example 1 was formulated as coated microparticles (as per U.S. Pat. No. 6,482,517 which is herein incorporated by reference), and shown in tests on rats that the formulation strongly increase the duration of action of bupivacaine. An amount 10.930 gm of Pluronic P123 was combined with 2.698 gm of free base bupivacaine, 10.912 gm of linalool, and 5.447 gm of sterile water, and stirred to form a reversed cubic phase. Of this, 24.982 grams of cubic phase was combined in a flask with 62.807 gm of a diethanolamine-N-acetyltryptophan solution; the latter was prepared by mixing 16.064 gm of diethanolamine, 36.841 gm of sterile water, and 22.491 gm of N-acetyltryptophan and sonicating to combine. The cubic phase/diethanolamine-NAT mixture was first shaken, then homogenized, and finally processed in a Microfluidics microfluidizer to a particle size less than 300 nm. While the material was still in the microfluidizer, 47.219 gm of a 25 wt % zinc acetate solution, and 5.377 gm of diethanolamine were added, and the total mixture microfluidized for 20 runs of 1.5 minutes each. Five ml of a hot (60 C) mixture of water and sorbitan monopalmitin (6%) was then injected during microfluidization, and next 5 ml of a 14% aqueous solution of albumin. After further microfluidizing, the dispersion was divided into 42 centrifuge tubes of 3.5 ml of dispersion each, and approximately 0.14 gm of Norit activated charcoal was added to each tube, and the tube shaken for 15 minutes on a rocker. Each tube was then centrifuged for 5 minutes in a 6000 rpm tabletop centrifuge. The dispersion was then prefiltered, then filtered at 0.8 microns using Millex AA filters, then placed in a sealed vial and shipped to a facility for animal testing.

The formulation was tested on male Spraque-Dawley rats, weighing 220-250 gm. The animals were maintained under standard conditions, with access to food and water ad libitum. They were briefly anesthetized with halothane to facilitate the injection. Sciatic nerve blockage was then tested by first making a small incision in the popliteal fossa space over the area of the sciatic nerve; the sciatic nerve was then visualized, identified, and the test agent or Marcaine control then injected into the sciatic nerve sheath and the incision closed surgically. Blockage of thermal nociception was determined by placing the rat on the glass surface of a thermal plantar testing apparatus (Model 336, IITC Inc.), with the surface maintained at 30 C. A mobile radiant heat source located under the glass was focused onto the hindpaw of the rat, and the paw-withdrawal latency recorded by digital timer. The baseline latency was found to be 10 seconds. The rats were tested for latency at 30 minutes and hourly thereafter.

The sensor blocking effect with the standard 0.5% bupivacaine HCl, at a dose of 3 mg/kg, was found to be 4-5 hours, in complete agreement with the well-known duration of Marcaine® nerve block. In contrast, at the same 3 mg/kg dose of the cubic phase formulation, the sensor blocking effect lasted approximately 22-26 hours. In addition, the latency time itself was greatly increased in the cubic phase case relative to the solution case, indicating a profound pain blockage. Drug efficacy was, therefore, not only undiminished but actually improved by the formulation. It is noted that while this dose of 3 mg/Kg was not super-toxic—and indeed, there were no deaths or serious sequellae—neither was is sub-toxic according to our definition above; that is, with respect to the latter, this would not be a dose that would fall within the recommended range of routine use. The relative duration in this Example was about 600% and the relative dose (based on a standard therapeutic dose of 1 mg/Kg) was 300%, making the amplification factor approximately 2.0.

### Example 3

While the previous Example used the excipient linalool—which is of very low toxicity but nonetheless not strictly pharmaceutically-acceptable for intravenous injection—and employed a fairly high dose of bupivacaine, 3 mg/Kg, the remaining Examples dealing with bupivacaine used lower doses of (1 mg/Kg or less), and alpha-tocopherol (Vitamin E) instead of linalool. Alpha-tocopherol is currently used in intravenous formulations for parenteral nutrition, and is thus pharmaceutically-acceptable for injection by the strict terms of the definition given above. Albumin and N-acetyltryptophan are both used in significant amounts in several intravenous human albumin formulations currently marketed, such as Plasbumin® and Buminate®, and indeed both are used at levels in excess of those levels that would be incurred in a 1 mg/Kg injection of the formulation in this Example, so these compounds are pharmaceutically-acceptable for injection as defined herein. Sorbitan monopalmitate appears on the 1996 FDA Inactive Ingredient List for injectable formulations. Working in a laminar flow hood, 0.900 grams of the local anesthetic bupivacaine, in its free base form, were dissolved in 3.64 gm of alpha-tocopherol (Aldrich Chemical Company, Milwaukee, Wis.) by heating to 55° C. Following dissolution, 1.820 gm of sterile water (Abbott Laboratories, Chicago, Ill.) and 3.640 gm of Pluronic P123 (BASF Corporation, Mt. Olive, N.J.) was added to the vitamin E. The components were mixed to form a reversed cubic phase that was optically isotropic and of high viscosity. Next, 0.402 gm of sodium deoxycholate (Aldrich Chemical Company, Milwaukee, Wis.) was dissolved in 39.6 ml of sterile water. An amount 8.048 gm of cubic phase was dispersed in the sodium deoxycholate solution, first using the homogenizer (Brinkmann Polytron PT 3000) at 29 k rpm for 1 minute, then using the microfluidizer (Micofluidics Model M110L) at approximately 15,000 psi for five 1.5 minute runs. The dispersion, referred to as “Lyotropic/F4C,” was injected into sterile vials using a 27 gauge needle attached to a 0.22 μm syringe filter (Millipore, Ireland).

Lyotropic/F4C was analyzed using a Beckman Coulter N4 PLUS submicron particle size analyzer. A drop of the dispersion was diluted in water until an adequate measurement intensity level was obtained. Essentially all of the particles in the dispersion are measured as less than 400 nm in size. Additionally, Lyotropic/F4C was analyzed using a Beckman Coulter DELSA 440SX for Doppler Electrophoretic Light Scattering Analysis, set in zeta potential measurement mode, using four angles of measurement. At all four angles, the distribution was centered at −31 mV, which is a strong enough zeta potential to produce a stable dispersion.

The above formulation was tested in the rat “Paw Withdrawal” model to determine the duration of analgesia. Male Spraque-Dawley rats, weighing 400-450 gm, were studied at two dose levels: 1.0 mg/kg and, for reference, 3.0 mg/kg. All rats were housed under standard conditions in accordance to AALAC guidelines, with access to food and water ad libitum. Six hours prior to evaluation, food was withheld.

PROCEDURE: Each rat was briefly anesthetized by exposure to the inhalational agent halothane in order to facilitate animal handling and to ensure precise injection of the test and control agents. Once unconscious, a small incision in the region of the popliteal fossa of the hind limb was made. Exposure of the sciatic nerve was obtained with minimal retraction. Utilizing an appropriately sized needle and syringe, either the bupivacaine-LyoCell® formulation or the standard bupivacaine hydrochloride was injected into the perineurium of the sciatic nerve. The incision was then closed with an appropriately sized surgical clip.

Local anesthetic blockade to thermal nociception was determined by exposure of the hind paw of the treated hind limb to the heated surface of a thermal plantar testing apparatus. Surface temperatures were maintained in a range from 50 to 54° C. The latency period to pay withdrawal from the heated surface was recorded by digital timer. Baseline latency period was found to be approximately 1 to 3 seconds in non-anesthetized hind paws. In an attempt to minimize thermal injury to the hind paw, maximum exposure to the thermal plantar testing apparatus was limited to 12 seconds. Latency periods exceeding 6 seconds were considered indicative of analgesia to thermal testing.

Six rats were tested for latency withdrawal of the treated hind limb after 30 minutes and 60 minutes, and then hourly for an additional five hours. At a dose of 1 mg/kg dose of the cubic phase formulation, the sensor blocking effect lasted over 5 hours, for 4 of the 6 rats tested and over 6 hours for two of the six rats tested.

### Example 4

The liquid crystalline dispersion containing the local anesthetic drug bupivacaine of Example 3 was prepared (“F4C”). The formulation was tested on male Sprague-Dawley rats, weighing 210-260 gms, in the rat “Paw Withdrawal” model of Example 3 at one dose level, 1.0 mg/kg, as was the standard bupivacaine hydrochloride solution (Marcaine® marketed by Astra-Zeneca). In order to avoid any bias from thermal trauma, test groups were evaluated in two segments:
Segment 1. Six rats were tested for latency withdrawal of the treated hind limb hourly for six hours.
Segment 2. If any animal(s) in Segment 1 exhibited continued analgesia to thermal testing at 6 hours, a 2nd group of six rats was injected and evaluated hourly on the thermal plantar testing apparatus at 16, 17 and 18 hours post administration. All rats were followed to normalization of latency periods to ensure that thermally induced nerve injury was not a factor in prolonged latency periods.
   The summary results are set forth in the three tables in Table Set 1. At every measurement time, the group administered F4C contained equal or more animals exhibiting nerve block than the group administered the standard solution. Beginning at 4 hours post administration, the number of animals in the standard solution group that were blocked dropped off significantly, while all animals in the F4C group remained blocked. This was also the case at 5 hours post administration. At 16 hours post administration, fully half the F4C group animals were blocked, and at 18 hours 2 of 6 animals in the F4C group were blocked. Because of the sharp drop off in animals blocked in the standard bupivacaine hydrochloride solution group (only 1 at 6 hours post administration), animals in this group were not tested at 16 through 18 hours. The relative duration in this Example was about [16 hrs]/[4 hrs]×100%=400%, and the relative dose 100%, making the amplification factor approximately 4.0.

Table Set 1

| | | | | | | | | |
|---|---|---|---|---|---|---|---|---|
| 1 hr | 2 hrs. | 3 hrs. | 4 hrs | 5 hrs | 6 hrs | 16 hrs | 17 hrs | 18 hrs |

SUMMARY: NUMBER OF BLOCKS

| | | | | | | | | | |
|---|---|---|---|---|---|---|---|---|---|
| F4C | 6 | 6 | 6 | 6 | 6 | 3 | 3 | 1 | 2 |
| Marcaine | 6 | 6 | 4 | 3 | 3 | 1 | NT | NT | NT |

SUMMARY: TOTAL SCORES (In Seconds)

| | | | | | | | | | |
|---|---|---|---|---|---|---|---|---|---|
| F4C | 71 | 71 | 66 | 72 | 65 | 49 | 43 | 31 | 33 |
| Marcaine | 69 | 66 | 51 | 44 | 40 | 31 | NT | NT | NT |

SUMMARY: AVERAGE SCORES

| | | | | | | | | | |
|---|---|---|---|---|---|---|---|---|---|
| F4C | 11.83 | 11.83 | 11.00 | 12.00 | 10.83 | 8.17 | 7.17 | 5.17 | 5.50 |
| Marcaine | 11.50 | 11.00 | 8.50 | 7.33 | 6.67 | 5.17 | NT | NT | NT |

NT = not tested

### Example 5

The liquid crystalline dispersion containing the local anesthetic drug bupivacaine of Example 3 was prepared (“F4C”). The formulation was tested on male Sprague-Dawley rats, weighing 200-275 gms, in the rat “Paw Withdrawal” model of Example 3 at three dose levels, 1.0 mg/kg, 0.67 mg/kg and 0.33 mg/kg, with six rats tested for each formulation for each dose. The standard bupivacaine hydrochloride solution (Marcaine®) also was tested at the same three dose levels. The test articles were supplied at a concentration of 1.5% active, and diluted as required with sterile water for injection to administer the 0.67 mg/kg and 0.33 mg/kg doses. The standard bupivacaine was supplied at a concentration of 0.75%, and diluted as required with sterile water for injection to administer the 0.33 mg/kg dose. The rats were tested for paw withdrawal latency at two hours after administration, and then beginning at four hours after administration every hour through eight hours after administration.

The summary results are set forth in the three tables in Table Set 2. At eight hours after administration, more than half the animals administered F4C, at all three dose levels, were experiencing sensor blocking effect, while none of the animals administered standard bupivacaine hydrochloride solution were (Table 1). In fact, none of the animals administered standard solution were blocked at 5 hours or after. This difference in effect between the F4C formulation and standard bupivacaine hydrochloride solution across dose groups is also manifest in the Total Scores (in Seconds) (Table 2) and the Average Score (Table 3): all animals administered F4C were blocked for a significantly longer duration than those administered standard solution at any of the administered doses. Thus, the animals administered F4C at 0.33 mg/kg exhibited significantly greater and longer blocking than the animals administered the standard solution, even at three times the dose. Furthermore, among the animals administered F4C, the two lower dose level groups exhibited significant sensor blocking effect. They also exhibited similar, if somewhat lower, total scores and average scores in comparison to the 1.0 mg/kg dose group, particularly when compared to the animals administered standard bupivacaine hydrochloride solution. The group administered F4C at the lowest test dose, 0.33 mg/kg, exhibited the same number of animals blocked as the group administered twice the dose (0.67 mg/kg), and exhibited even higher total scores and average scores than the 0.67 mg/kg group.

Table Set 2

| | | | | | |
|---|---|---|---|---|---|
| 2 hrs | 4 hrs | 5 hrs | 6 hrs | 7 hrs | 8 hrs |

SUMMARY: NUMBER OF BLOCKS

| | | | | | | |
|---|---|---|---|---|---|---|
| F4C 0.33 | 6 | 6 | 6 | 5 | 5 | 4 |
| F4C 0.67 | 6 | 6 | 6 | 4 | 4 | 4 |
| F4C 1.0 | 6 | 6 | 6 | 6 | 6 | 6 |
| Marcaine 0.33 | 4 | 0 | 0 | 0 | 0 | 0 |
| Marcaine 0.67 | 6 | 3 | 0 | 0 | 0 | 0 |
| Marcaine 1.0 | 6 | 5 | 0 | 0 | 0 | 0 |

SUMMARY: TOTAL SCORES (In Seconds)

| | | | | | | |
|---|---|---|---|---|---|---|
| F4C 0.33 | 69 | 66 | 65 | 61 | 57 | 53 |
| F4C 0.67 | 70 | 61 | 56 | 49 | 46 | 46 |
| F4C 1.0 | 70 | 69 | 66 | 64 | 61 | 59 |
| Marcaine 0.33 | 46 | 24 | 0 | 0 | 0 | 0 |
| Marcaine 0.67 | 69 | 37 | 25 | 0 | 0 | 0 |
| Marcaine 1.0 | 61 | 48 | 30 | 24 | 0 | 0 |

SUMMARY: AVERAGE SCORES

| | | | | | | |
|---|---|---|---|---|---|---|
| F4C 0.33 | 11.50 | 11.00 | 10.83 | 10.17 | 9.50 | 8.83 |
| F4C 0.67 | 11.67 | 10.17 | 9.33 | 8.17 | 7.67 | 7.67 |
| F4C 1.0 | 11.67 | 11.50 | 11.00 | 10.67 | 10.17 | 9.83 |
| Marcaine 0.33 | 7.67 | 4.00 | 0 | 0 | 0 | 0 |
| Marcaine 0.67 | 11.50 | 6.17 | 4.17 | 0 | 0 | 0 |
| Marcaine 1.0 | 10.17 | 8.00 | 5.00 | 4.00 | 0 | 0 |

### Example 6

An amount 15.027 gm of Pluronic P123 was combined with 2.703 gm of free base bupivacaine, 10.972 gm of tocepherol (Vitamin E), and 5.464 gm of sterile water, and stirred to form a reversed cubic phase. Of this, 25.018 grams of cubic phase was combined in a flask with 62.872 gm of a diethanolamine-N-acetyltryptophan solution; the latter was prepared by mixing 16.037 gm of diethanolamine, 36.838 gm of sterile water, and 22.5031 gm of N-acetyltryptophan and sonicating to combine. The cubic phase/diethanolamine-NAT mixture was first shaken, then homogenized, and finally processed in a Microfluidics microfluidizer to a particle size less than 300 nm. While the material was still in the microfluidizer, 47.279 gm of a 25 wt % zinc acetate solution, and 5.371 gm of diethanolamine were added, and the total mixture microfluidized for 21 runs of 1.5 minutes each. Five ml of a hot (60 C) mixture of water and sorbitan monopalmitin (6%) was then injected during microfluidization, and next 5 ml of a 15% aqueous solution of albumin. After further microfluidizing, the dispersion was divided into centrifuge tubes of 3.5 ml of dispersion each, and approximately 0.14 gm of Norit activated charcoal was added to each tube, and the tube shaken for 15 minutes on a rocker. Each tube was then centrifuged for 5 minutes in a 6000 rpm tabletop centrifuge. The dispersion was then prefiltered, then filtered at 0.8 microns using Millex AA filters, then placed in a sealed vial and shipped to a facility for animal testing. This formulation was referenced as “F2V”.

This formulations was tested on male Sprague-Dawley rats in the “Paw Withdrawal” model to determine the duration of analgesia. Male Sprague-Dawley rats, weighing 200-260 gm were studied at one dose level, 1.0 mg/kg. Surface temperatures were maintained in a range from 50 to 54 degree C. The latency period to pay withdrawal from the heated surface was recorded by digital timer. Baseline latency period was found to be approximately 1 to 3 seconds in non-anesthetized hind paws. In an attempt to minimize thermal injury to the hind paw, maximum exposure to the thermal plantar testing apparatus was limited to 12 seconds. Latency periods exceeding 6 seconds were considered indicative of analgesia to thermal testing. Six rats comprised each group, and were tested for paw withdrawal latency of the treated hind limb every hour beginning at one hour post administration and continuing through six hours post administration. In order to avoid any bias from thermal trauma, test groups were evaluated in two segments, as described above.

The summary results are set forth in the three tables in Table Set 3. At every measurement time, all of the groups administered F2V contained equal or more animals exhibiting nerve block than the groups administered the standard bupivacaine hydrochloride solution. Beginning at 4 hours post administration, the number of animals in the standard bupivacaine hydrochloride solution group that were blocked dropped off significantly, while all animals in F2V groups remained blocked. This was also the case at 5 hours post administration, and continued to be the case for the F2V group at 6 hours post administration. At 16 hours post administration, more than half of the F2V group was blocked. At 17 hours 5 of the six animals in the F2V group were blocked. Because of the sharp drop off in animals blocked in the standard bupivacaine hydrochloride solution group (only 1 at 6 hours post administration), animals in this group were not tested at 16 through 18 hours. Total Scores (in seconds) and Average scores for each group are consistent, and show significantly higher scores for the F2V group than the standard bupivacaine hydrochloride solution at five hours post administration and after. The relative duration in this Example was about [17 hrs]/[4 hrs]×100%=425%, and the relative dose 100%, making the amplification factor approximately 4.25.

Table Set 3

| | | | | | | | | |
|---|---|---|---|---|---|---|---|---|
| 1 hr | 2 hrs | 3 hrs | 4 hrs | 5 hrs | 6 hrs | 16 hrs | 17 hrs | 18 hrs |

SUMMARY: NUMBER OF BLOCKS

| | | | | | | | | | |
|---|---|---|---|---|---|---|---|---|---|
| F2V | 6 | 6 | 6 | 6 | 6 | 6 | 4 | 5 | 1 |
| Marcaine | 6 | 6 | 4 | 3 | 3 | 1 | NT | NT | NT |

SUMMARY: TOTAL SCORES (In Seconds)

| | | | | | | | | | |
|---|---|---|---|---|---|---|---|---|---|
| F2V | 72 | 71 | 68 | 71 | 70 | 64 | 53 | 56 | 37 |
| Marcaine | 69 | 66 | 51 | 44 | 40 | 31 | NT | NT | NT |

SUMMARY: AVERAGE SCORES

| | | | | | | | | | |
|---|---|---|---|---|---|---|---|---|---|
| F2V | 12.00 | 11.83 | 11.33 | 11.83 | 11.67 | 10.67 | 8.83 | 9.33 | 6.17 |
| Marcaine | 11.50 | 11.00 | 8.50 | 7.33 | 6.67 | 5.17 | NT | NT | NT |

NT = not tested

### Example 7

The coated particle liquid crystalline dispersion containing the local anesthetic drug bupivacaine of Example 6 was prepared (“F2V”). The formulation was tested on male Sprague-Dawley rats, weighing 200-275 gms, in the rat “Paw Withdrawal” model of Example 103 at three dose levels, 1.0 mg/kg, 0.67 mg/kg and 0.33 mg/kg, with six rats tested for each formulation for each dose. The standard bupivacaine hydrochloride solution (Marcaine®) also was tested at the same three dose levels. The test articles were supplied at a concentration of 1.5% active, and diluted as required with sterile water for injection to administer the 0.67 mg/kg and 0.33 mg/kg doses. The standard bupivacaine was supplied at a concentration of 0.75%, and diluted as required with sterile water for injection to administer the 0.33 mg/kg dose. The rats were tested for paw withdrawal latency at two hours after administration, and then beginning at four hours after administration every hour through seven hours after administration.

The summary results are set forth in the three tables in Table Set 4. At seven hours after administration, more than half the animals administered F2V, at all three dose levels, were experiencing sensor blocking effect, while none of the animals administered standard bupivacaine hydrochloride solution were (Table 1 of Table Set 4). In fact, none of the animals administered standard bupivacaine hydrochloride solution was blocked at 6 hours or after. This difference in effect between the F2V formulation and standard bupivacaine hydrochloride solution across dose groups is also manifest in the Total Scores (in Seconds) (Table 2 of Table Set 4) and the Average Score (Table 3 of Table Set 4): all animals administered F2V were blocked for a significantly longer duration than those administered standard bupivacaine hydrochloride solution at any of the administered doses. Thus, the animals administered F2V at 0.33 mg/kg exhibited significantly greater and longer blocking than the animals administered the standard bupivacaine hydrochloride solution, even at three times the dose. Furthermore, among the animals administered F2V, the two lower dose level groups exhibited significant sensor blocking effect. They also exhibited similar or greater total scores and average scores in comparison to the 0.67 mg/kg and 1.0 mg/kg dose group, particularly when compared to the animals administered standard bupivacaine hydrochloride solution. The group administered F2V at the lowest test dose, 0.33 mg/Kg, exhibited the same number of animals blocked as the group administered twice the dose (0.67 mg/Kg) and one more than the group administered three times the dose (1.0 mg/Kg).

Focusing on the results at 0.33 mg/Kg, we note that the duration of action was more than 7 hours (the maximum time allowed due to experimental constraints), since 5 of 6 rats were still blocked after 7 hours. This allows us to put a lower limit on the amplification factor. Using this 7 hour figure, the relative duration in this Example was [7 hrs]/[4 hrs]×100%=175%, and the relative dose 33%, making the amplification factor approximately 5.25.

Table Set 4

| | | | | | | |
|---|---|---|---|---|---|---|
| 1 hr | 2 hrs | 3 hrs | 4 hrs | 5 hrs | 6 hrs | 7 hrs |

SUMMARY: NUMBER OF BLOCKS

| | | | | | | | |
|---|---|---|---|---|---|---|---|
| F2V 0.33 | 6 | 6 | 6 | 5 | 5 | 5 | 5 |
| F2V 0.67 | 6 | 6 | 6 | 6 | 6 | 5 | 5 |
| F2V 1.0 | 6 | 6 | 6 | 6 | 5 | 5 | 4 |
| Marcaine 0.33 | 6 | 6 | 3 | 2 | 0 | 0 | NT |
| Marcaine 0.67 | 6 | 6 | 1 | 0 | 1 | 0 | NT |
| Marcaine 1.0 | 6 | 5 | 5 | 3 | 3 | 0 | NT |

SUMMARY: TOTAL SCORES (In Seconds)

| | | | | | | | |
|---|---|---|---|---|---|---|---|
| F2V 0.33 | 70 | 66 | 58 | 61 | 57 | 58 | 55 |
| F2V 0.67 | 72 | 63 | 72 | 67 | 66 | 55 | 56 |
| F2V 1.0 | 72 | 63 | 70 | 65 | 60 | 54 | 52 |
| Marcaine 0.33 | 72 | 54 | 46 | 36 | 28 | 28 | NT |
| Marcaine 0.67 | 72 | 58 | 33 | 27 | 29 | 24 | NT |
| Marcaine 1.0 | 68 | 53 | 52 | 46 | 39 | 24 | NT |

SUMMARY: AVERAGE SCORES

| | | | | | | | |
|---|---|---|---|---|---|---|---|
| F2V 0.33 | 11.67 | 11.00 | 9.67 | 10.17 | 9.50 | 9.67 | 9.17 |
| F2V 0.67 | 12.00 | 10.50 | 12.00 | 11.17 | 11.00 | 9.17 | 9.33 |
| F2V 1.0 | 12.00 | 10.50 | 11.67 | 10.83 | 10.00 | 9.00 | 8.67 |
| Marcaine 0.33 | 12.00 | 9.00 | 7.67 | 6.00 | 4.67 | 4.67 | NT |
| Marcaine 0.67 | 12.00 | 9.67 | 5.50 | 4.50 | 4.83 | 4.00 | NT |
| Marcaine 1.0 | 11.33 | 8.83 | 8.67 | 7.67 | 6.50 | 4.00 | NT |

NT = Not Tested

### Example 8

In this example, the anticancer drug paclitaxel was solubilized in a Pluronic-essential oil-water cubic phase, which was encapsulated by a zinc-NAT shell as in Example 2. The cubic phase was prepared by mixing 0.070 gm of gum benzoin, 0.805 gm of essential oil of sweet basil, and 0.851 gm of oil of ylang-ylang, heating to dissolve the gum benzoin, then adding 265 mg of paclitaxel, 3.257 gm of oil of spearmint, 0.640 gm of strawberry aldehyde, 0.220 gm of ethylhexanoic acid, 1.988 gm of deionized water, and finally 3.909 gm of Pluronic 103. The encapsulating with zinc-NAT was done similarly as in the previous Example, except that short homogenizing was used instead of microfluidizing. No monopalmitin was incorporated, and the Norit charcoal purification step was omitted skipped. The dispersion was placed in vials and sent for testing oral absorption in dogs.

Beagle dogs, 10-12 kg in weight, were cannulated to allow delivery of the formulation directly into the duodenum. Paclitaxel is known to exhibit very low absorption given orally or intraduodenally. Indeed, even in the Taxol^{R} formulation, which includes a large volume of surfactant (Cremophor EL) and ethanol, both of which are membrane fluidizers, the bioavailability is less than about 10%.

Blood levels of paclitaxel were measured at predose, 20 minutes, 40 minutes, 1 hour, 2 hours, 3 hours, 4 hours, 8 hours, 10 hours, and 24 hours. The results for one experiment with the cubic phase formulation were as follows:

| Time point | | Blood concentration (ng/ml) |
|---|---|---|
| 20 | min | 79.4 |
| 40 | min | 149 |
| 1 | hour | 122 |
| 2 | hour | 100 |
| 3 | hour | 79.5 |
| 4 | hour | 70.1 |
| 8 | hour | 43.2 |
| 10 | hour | 31.1 |
| 24 | hour | 17.6 |

These blood levels, which are extended over many hours, indicate a high degree of absorption of paclitaxel and sustained systemic levels, and thus a very strong enhancement of efficacy due to the cubic phase vehicle in which the paclitaxel was dissolved. As a comparison, U.S. Pat. No. 6,730,698 to Broder et al. shows results of oral administration in rats of 9 mg/Kg—that is, 9-fold higher dose than used in this current Example—where maximum blood levels of about 30 ng/ml were reached, and after only 4 hours blood levels were down to less than 10 ng/ml. If we take relative duration of drug action to be roughly (and fairly conservatively, one could argue) given by [24 hrs]/[4 hrs]×100%=600%, and the relative dose to be [1 mg/Kg]/[9 mg/Kg]=11%, then the amplification factor here is about 600%/11%=54.0. While this dramatic result was an early-stage result and should not be taken as a consistently reproducible result, it does give an indication as to the potential inherent in the formulations of this invention in the realm of oral drug delivery. Paclitaxel is of course well known to exhibit significant systemic dose-dependent toxicities.

1-145. (canceled)

146. A local anesthetic formulation having prolonged duration of action for a given dose, comprising:
a local anesthetic selected from the group consisting of bupivacaine and ropivacaine at a given dose;
microparticles of reversed cubic or reversed hexagonal lyotropic liquid crystalline phase material or combinations thereof ; and
a polar solvent,
wherein said microparticles are dispersed in said polar solvent,
wherein said local anesthetic is solubilized in said microparticles,
wherein said given dose of said local anesthetic has a first duration of action in the absence of said microparticles and said polar solvent, and has a second duration of action which is greater than said first duration of action when said local anesthetic is solubilized in said microparticles and said microparticles are dispersed in said polar solvent.

147. The local anesthetic formulation of claim 146 wherein said local anesthetic formulation produces a duration of nerve block of at least eight hours in a rat paw withdrawal nerve block model when said given dose is an amount equal to a standard therapeutic dose.

148. The local anesthetic formulation of claim 146 wherein said local anesthetic formulation does not include a vasoconstrictive agent.

149. The local anesthetic formulation of claim 146 wherein said microparticles are uncoated and a zeta potential of said microparticles in said formulation equals or exceeds 25 millivolts in magnitude.

150. The local anesthetic formulation of claim 146 wherein said microparticles are coated.

151. The local anesthetic formulation of claim 146 wherein said microparticles have an average size of less than 1.0 micron.

152. The local anesthetic formulation of claim 146 wherein said microparticles have an average size of less than 0.5 micron.

153. The local anesthetic of claim 146 wherein said second duration of action is at least 50% greater than said first duration of action.

154. A method of providing prolonged anesthesia to a subject, comprising administering to said subject a local anesthetic formulation having
a local anesthetic selected from the group consisting of bupivacaine and ropivacaine at a given dose;
microparticles of reversed cubic or reversed hexagonal lyotropic liquid crystalline phase material or combinations thereof ; and
a polar solvent,
wherein said microparticles are dispersed in said polar solvent,
wherein said local anesthetic is solubilized in said microparticles,
wherein said given dose of said local anesthetic has a first duration of action in the absence of said microparticles and said polar solvent, and has a second duration of action which is greater than said first duration of action when said local anesthetic is solubilized in said microparticles and said microparticles are dispersed in said polar solvent.

155. The method of claim 153 wherein said step of administering is performed by injection.

156. The method of claim 153 wherein said step of administering is performed by spraying or nebulization.
